(19) United States
(12) Patent Application Publication
(10) Pub. No.: US 2023/0334520 A1
(43) Pub. Date: Oct. 19, 2023

(54) INFORMATION PROCESSING METHOD, INFORMATION PROCESSING DEVICE, AND NON-TRANSITORY COMPUTER READABLE RECORDING MEDIUM

(71) Applicant: Panasonic Intellectual Property Corporation of America, Torrance, CA (US)

(72) Inventors: Kotaro SAKATA, Osaka (JP); Tetsuji FUCHIKAMI, Osaka (JP)

(21) Appl. No.: 18/212,802

(22) Filed: Jun. 22, 2023

Related U.S. Application Data
(63) Continuation of application No. PCT/JP2021/041415, filed on Nov. 10, 2021.

(30) Foreign Application Priority Data
Dec. 28, 2020 (JP) 2020-218797

(51) Int. Cl. G06Q 30/0207; G06N 3/126; G16B 20/20; G16B 40/20

(52) U.S. Cl. CPC G06Q 30/0207; G06N 3/126; G16B 20/20; G16B 40/20

(57) ABSTRACT

An information processing device includes: an acquisition part that acquires genetic data detected by a gene detector and including a base sequence indicating a genotype of a user; a region specifying part that specifies a region where the genetic data is located in the reference data; a rarity degree calculation part that calculates a rarity degree indicating a rarity of the genetic data on the basis of a data density associated with the specified region; an incentive calculation part that calculates an incentive to be given to the user in accordance with the calculated rarity degree; and an output part that outputs the calculated incentive.

### TECHNICAL FIELD

The present disclosure relates to a technology of collecting genetic data.

### BACKGROUND ART

A technology called the SNP (single nucleotide polymorphism) genotype imputation has been known in recent years as a technology of estimating a genotype of a region which is not acquirable with an SNP microarray. The SNP genotype imputation uses reference data including information indicating an SNP genotype in a high density. Effective collection of genetic data of a region having a low density, i.e., effective collection of genetic data having a rarity, rather than random collection of the genetic data is demanded to establish the reference data having the high density.

Patent Literature 1 discloses a method of providing, by utilizing a blockchain technology, bio-information data to prevent exposure of bio-information data, and avoid forgery or tampering of genomic data.

Patent Literature 2 discloses an information transaction device that provides an information user with only user information about an information provider whose agreement is made on a reward offered to the information provider, and adjusts the reward in accordance with an acquisition situation of the user information.

However, neither of the conventional technologies described above considers effectively collecting genetic data having a rarity, and thus needs further improvement.

### CITATION LIST

### Patent Literature

Patent Literature 1: Japanese Patent Publication No. 6661742
Patent Literature 2: Japanese Patent Publication No. 5978198

### SUMMARY OF INVENTION

The present disclosure has been achieved to solve the aforementioned drawbacks, and has an object of providing a technology of effectively collecting genetic data having a rarity.

An information processing method according to one aspect of this disclosure is an information processing method for an information processing device that performs an information process by using reference data. The information processing method includes: acquiring genetic data detected by a gene detector and including a base sequence indicating a genotype of a user; specifying a region where the genetic data is located in the reference data, the reference data being data that in which a base sequence indicating a genotype of a genome is associated in advance with a data density according to a locus of the base sequence; calculating, on the basis of the data density associated with the specified region, a rarity degree indicating a rarity of the genetic data; calculating an incentive to be given to the user in accordance with the calculated rarity degree; and outputting the calculated incentive.

This disclosure achieves effective collection of genetic data having a rarity.

### BRIEF DESCRIPTION OF DRAWINGS

FIG. 1 is a diagram showing an example of an overall configuration of an information processing system adopting an information processing device in a first embodiment of the present disclosure.

FIG. 2 is a block diagram showing an example of a configuration of the information processing device shown in FIG. 1.

FIG. 3 is an explanatory view of terms related to a genetic analysis.

FIG. 4 illustrates an example of a data configuration of reference data.

FIG. 5 is an illustration expressing the reference data in accordance with a data density.

FIG. 6 is a flowchart showing an example of a process by the information processing device in the first embodiment of the disclosure.

FIG. 7 is a block diagram showing an example of a configuration of an information processing device in a second embodiment of the disclosure.

FIG. 8 illustrates an example of a data configuration of area reference data.

FIG. 9 is an illustration expressing the area reference data illustrated in FIG. 8 in accordance with the data density.

FIG. 10 is a flowchart showing an example of a process by the information processing device in the second embodiment of the disclosure.

### DESCRIPTION OF EMBODIMENTS

Circumstances Led Up to this Disclosure

Researches concerning the genome-wide association study for evaluating an association of a target trait with an SNP genotype have been developed through genotyping on hundreds of thousands of people to specify the SNP genotypes in tens of millions of portions that cover the whole human genomes. The genome-wide association study needs tens of millions of SNP genotypes. Meanwhile, an SNP microarray has been widely used to facilitate the genotyping for the SNP at a low cost in recent years.

With the SNP microarray, only SNP genotypes in hundreds of thousands of portions are acquirable, and thus, genetic data obtained with the SNP microarray is not directly applicable to the genome-wide association study. Under the circumstances, the SNP genotype imputation is employed to statistically estimate tens of millions of SPN genotypes from the genetic data obtained with the SNP microarray.

In the SNP genotype imputation, a base sequence in reference data complements a base sequence in the genetic data obtained with the SNP microarray to estimate a genotype of the SNP in an unobserved region. However, reference data having a high density of SNP genotype is required for execution of the SNP genotype imputation. For this purpose, effective collection of genetic data of a region having a low density, that is, effective collection of genetic data having a rarity, is demanded rather than random collection of genetic data.

Patent Literature 1 merely discloses providing, by utilizing the blockchain technology, bio-information data of a second user encrypted by a public key of the second user to a first user having succeeded in user authentication. An object of the patent literature is only to prevent the exposure of the bio-information data, and prevent forgery or tampering of the genomic data. Patent Literature 1 hence cannot effectively collect genetic data having a rarity.

Patent Literature 2 discloses user information provided by an information provider, the user information including: positional information; atmospheric information; sound acquisition information; illuminance information; frequency information; and personal information including an age, an occupation, and an annual income, but fails to disclose genetic data. Patent Literature 2 thus cannot determine an appropriate incentive to be given to the information provider in accordance with a rarity of genetic data, resulting in a failure to effectively collect genetic data having a rarity.

The present inventors taking this into consideration have conceived of respective aspects of the present disclosure as described below to effectively collect genetic data having a rarity.

An information processing method according to one aspect of the present disclosure is an information processing method for an information processing device that performs an information process by using reference data. The information processing method includes: acquiring genetic data detected by a gene detector and including a base sequence indicating a genotype of a user; specifying a region where the genetic data is located in the reference data, the reference data being data that in which a base sequence indicating a genotype of a genome is associated in advance with a data density according to a locus of the base sequence; calculating, on the basis of the data density associated with the specified region, a rarity degree indicating a rarity of the genetic data; calculating an incentive to be given to the user in accordance with the calculated rarity degree; and outputting the calculated incentive.

According to this configuration, a region where the genetic data provided from the user is located in the reference data is specified, and a rarity degree of the genetic data is calculated on the basis of a data density associated with the specified region. An incentive to be given to the user in accordance with the rarity degree is then calculated and the calculated incentive is output. The configuration succeeds in giving a higher incentive to a user having provided genetic data having a higher rarity degree than an incentive to another user having provided genetic data having a lower rarity degree. This results in achieving effective collection of the genetic data having a rarity.

In the information processing method, the genetic data may be associated with attribute information including an attribute of the user. The information processing method may further include calculating, on the basis of the attribute information, a contribution degree of the genetic data to a genetic analysis. In the calculating of the incentive, the incentive may be calculated in accordance with the rarity degree and the contribution degree.

Adopting such attribute information about the user having provided the genetic data in the genetic analysis using the genetic data increases the possibility of obtaining a useful genetic analysis result. According to this configuration, the contribution degree to the genetic analysis is calculated on the basis of the attribute information, and the incentive is calculated in further consideration of the calculated contribution degree. This configuration thus motivates the user to provide the attribute information, resulting in achieving effective collection of the genetic data associated with the useful attribute information.

In the information processing method, the genetic data may be associated with locus information indicating a locus of the base sequence indicating the genotype, and, in the calculating of the rarity degree, the region where the genetic data may be located in the reference data is specified on the basis of the locus information.

This configuration associates genetic data with the locus information indicating a locus of a gene, and thus facilitates specifying of the region where the genetic data is located in the reference data.

In the information processing method, the attribute information may include information indicating a residence of the user, the reference data may include a plurality of pieces of area reference data respectively for predetermined areas, and, in the specifying of the region, the region where the genetic data is located in area reference data corresponding to the information about the residence may be specified.

Genotypes of users who live in the same area have a similar tendency, and thus execution of the SNP genotype imputation by using area reference data for the area increases an estimation accuracy. In this case, genetic data of a user living in a residence defined as an area corresponding to area reference data having a low data density has a higher rarity than genetic data of another user living in a residence defined as an area corresponding to area reference data having a high data density. The configuration enables calculation of an incentive in accordance with the residence of the user having provided the genetic data. This consequently achieves effective collection of genetic data having a rarity in terms of an area.

In the information processing method, in the calculating of the contribution degree, the attribute information may be determined whether to include information indicating a blood relation of the user, and the contribution degree may be calculated to be higher in determination that the information indicating the blood relation is included than in determination that the information indicating the blood relation is not included.

This configuration succeeds in giving a higher incentive to a user when the attribute information includes the blood relation of the user. The configuration thus motivates the user to provide the information indicating the blood relation which is available in a genetic analysis, resulting in achieving effective collection of the information indicating the blood relation.

In the information processing method, in the calculating of the contribution degree, the contribution degree may be calculated to be higher as an information amount of the information indicating the blood relation in the attribute information becomes greater.

This configuration succeeds in giving a higher incentive to the user when an information amount of the information indicating the blood relation becomes greater. The configuration thus achieves effective collection of the information indicating the blood relation with satisfactory contents.

In the information processing method, in the calculating of the contribution degree, the attribute information may be determined whether to include information indicating a life pattern of the user, and the contribution degree may be calculated to be higher in determination that the information indicating the life pattern is included than in determination that the information indicating the life pattern is not included.

This configuration succeeds in giving a higher incentive to a user when the attribute information includes the life pattern information about the user. The configuration thus motivates the user to provide the information indicating life pattern data which is available in a research of epigenetics, resulting in achieving effective collection of the life pattern data.

In the information processing method, in the calculating of the contribution degree, the contribution degree may be calculated to be higher as an information amount of the information indicating the life pattern of the user in the attribute information becomes greater.

This configuration succeeds in giving a higher incentive to the user when an information amount of the information indicating the life pattern becomes greater. The configuration thus achieves effective collection of the information indicating the life pattern with satisfactory contents.

An information processing device according to another aspect of the present disclosure is an information processing device that performs an information process by using reference data. The information processing device includes:
an acquisition part that acquires genetic data detected by a gene detector and including a base sequence indicating a genotype of a user; a region specifying part that specifies a region where the genetic data is located in the reference data, the reference data being data that in which a base sequence indicating a genotype of a genome is associated in advance with a data density according to a locus of the base sequence; a rarity degree calculation part that calculates a rarity degree indicating a rarity of the genetic data on the basis of the data density associated with the region specified by the region specifying part; an incentive calculation part that calculates an incentive to be given to the user in accordance with the rarity degree calculated by the rarity degree calculation part; and an output part that outputs the incentive calculated by the incentive calculation part.

An information processing program according to still another aspect of the present disclosure is an information processing program causing a computer to serve as an information processing device that performs information processing by using reference data. The information processing program includes: causing the computer to further serve as: an acquisition part that acquires genetic data detected by a gene detector and including a base sequence indicating a genotype of a user; a region specifying part that specifies a region where the genetic data is located in the reference data, the reference data being data that in which a base sequence indicating a genotype of a genome is associated in advance with a data density according to a locus of the base sequence; a rarity degree calculation part that calculates a rarity degree indicating a rarity of the genetic data on the basis of the data density associated with the region specified by the region specifying part; an incentive calculation part that calculates an incentive to be given to the user in accordance with the rarity degree calculated by the rarity degree calculation part; and an output part that outputs the incentive calculated by the incentive calculation part.

This disclosure can be realized as an information processing system caused to operate by the information processing program as well. Additionally, it goes without saying that the information processing program is distributable as a non-transitory computer readable storage medium like a CD-ROM, or distributable via a communication network like the Internet.

Each of the embodiments which will be described below represents a specific example of the disclosure. Numeric values, shapes, constituent elements, steps, and the order of the steps described below in each embodiment are mere examples, and thus should not be construed to delimit the disclosure. Moreover, constituent elements which are not recited in the independent claims each showing the broadest concept among the constituent elements in the embodiments are described as selectable constituent elements. The respective contents are combinable with each other in all the embodiments.

### First Embodiment

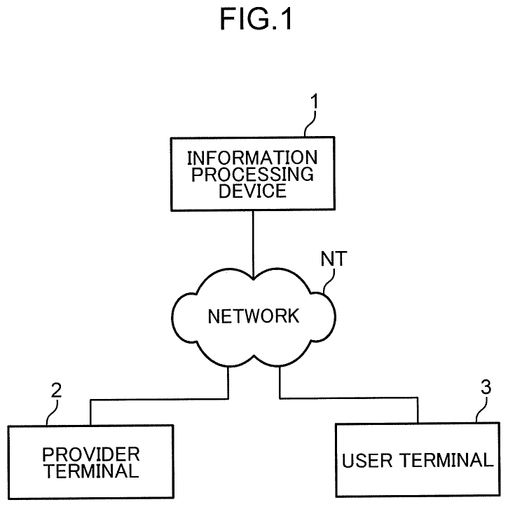

FIG. 1 is a diagram showing an example of an overall configuration of an information processing system adopting an information processing device 1 in a first embodiment of the present disclosure. The information processing system includes the information processing device 1, a provider terminal 2, and a user terminal 3. The information processing device 1 to the user terminal 3 are communicably connected to one another via a network NT.

The information processing device 1 includes, for example, a cloud server including one or more computers. The information processing device 1 receives genetic data provided by a user from the provider terminal 2, and calculates an incentive to be given to the user on the basis of the received genetic data.

The provider terminal 2 includes a computer, for example, owned by a medical institution to transmit genetic data to the information processing device 1. The genetic data is detected by a gene detector and includes a base sequence indicating a genotype of the user. For instance, the SNP microarray is adoptable as the gene detector. The SNP microarray includes DNA fragments arranged on a tip in a high density, each fragment working as a probe to detect a difference between base sequences. The SNP microarray detects SNP genotypes in hundreds of thousands of portions. The gene detector is not limited to the SNP microarray, and another device or component may be adopted.

Genetic data is associated with a user identifier identifying the user who provides the genetic data. The genetic data is further associated with locus information indicating a locus of a base sequence indicating an SNP genotype. The locus information indicates the locus on a genome of the base sequence indicating the SNP genotype.

The user terminal 3 serves as an information processing device owned by the user who provides the genetic data. In detail, the user terminal 3 includes a personal digital assistance, e.g., a smartphone and a tablet terminal, or a stationary computer like a laptop computer. The user terminal 3 acquires attribute information input by the user and transmits the acquired attribute information to the information processing device 1.

The network NT includes, for example, a wide area network having the internet and a mobile phone communication network.

The genetic data here is transmitted from the provider terminal 2 to the information processing device 1, but the present disclosure is not limited thereto. The genetic data may be transmitted from the user terminal 3 to the information processing device 1. In this case, the user terminal 3 may acquire genetic data detected by the SNP microarray, and transmit the genetic data to the information processing device 1 in association with the attribute information. Alternatively, the attribute information may be transmitted from the provider terminal 2. In this case, the provider terminal 2 may acquire the genetic data detected by the SNP microarray, and transmit the genetic data to the information processing device 1 in association with the attribute information.

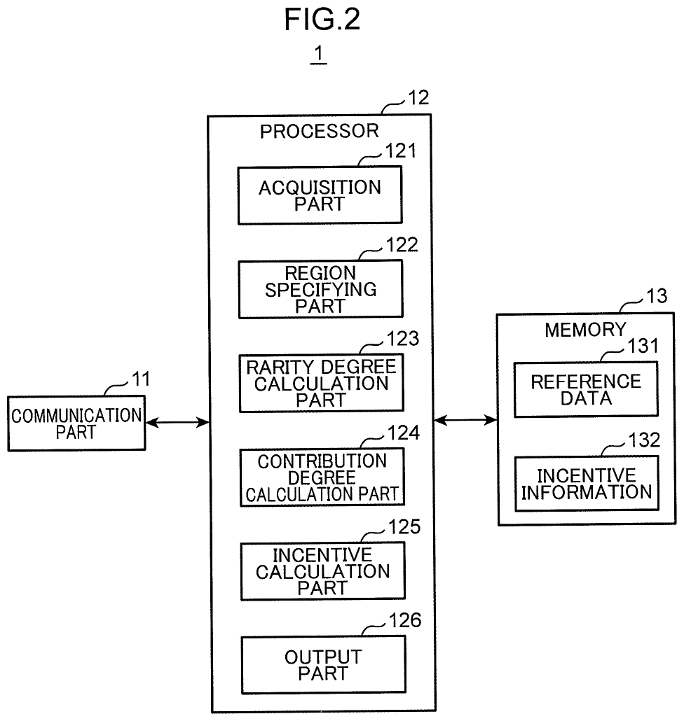

FIG. 2 is a block diagram showing an example of a configuration of the information processing device 1 shown in FIG. 1. The information processing device 1 includes a communication part 11, a processor 12, and a memory 13. The communication part 11 includes a communication circuit for connecting the information processing device 1 to the network NT. The communication part 11 receives the genetic data transmitted from the provider terminal 2. The genetic data received here is associated with the user identifier and the locus information. The communication part 11 receives the attribute information transmitted from the user terminal 3. The received attribute information is associated with the user identifier.

The memory 13 includes a non-volatile storage device, such as, an SSD (Solid State Drive) or an HDD (Hard Disc Drive). The memory 13 stores reference data 131 and incentive information 132.

The reference data 131 is used in the genotype imputation, and a base sequence indicating a genotype of a genome of a person is associated with a data density according to a locus of the base sequence in the reference data.

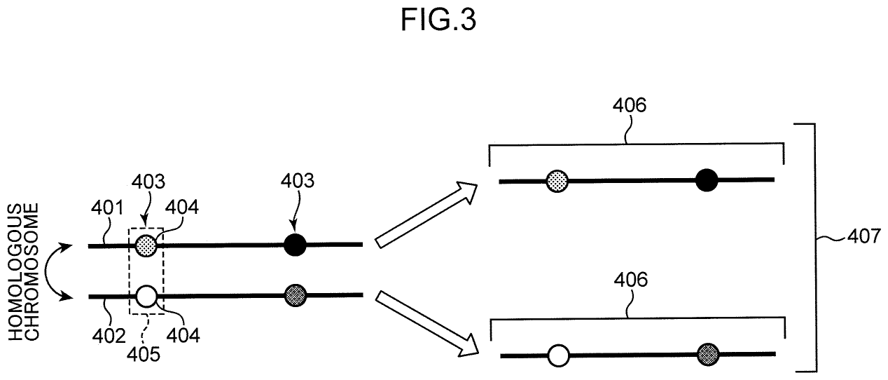

Here, terms used for a genetic analysis will be described. FIG. 3 is an explanatory view of terms related to the genetic analysis. FIG. 3 shows homologous chromosomes 401, 402 respectively denoted by two straight lines. A locus 403 indicates a location of a gene on each of the homologous chromosomes 401, 402. Alleles 404 designate genes forming a pair respectively on the homologous chromosomes 401, 402. A genotype 405 designates a combination of alleles 404. A haplotype 406 designates a combination of alleles 404. A diplotype 407 designates a combination of haplotypes 406.

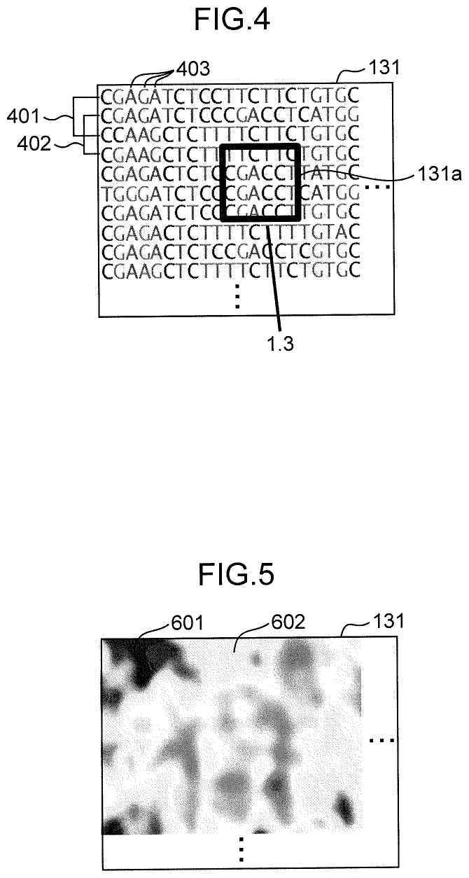

Next, a specific example of the reference data 131 will be described. FIG. 4 illustrates an example of a data configuration of the reference data 131. In the example in FIG. 4, the reference data 131 has a data structure in which two base sequences for each of the homologous chromosomes 401, 402 are arranged to alternately meander in respectively two rows. For instance, the base sequence of the homologous chromosome 401 is disposed in the first row, the base sequence of the homologous chromosome 402 is disposed in the second row, the base sequence of the homologous chromosome 401 continuous from the first row is disposed in the third row, and the base sequence of the homologous chromosome 402 continuous from the second row is disposed in the fourth row.

Besides, each locus 403 of the base sequence in the reference data 131 is associated with a data density. The data density has a value determined in accordance with the number of data pieces used to decide a base sequence at a certain locus 403. For instance, the data density is set to a higher value in accordance with an increase in the number of data pieces in such a manner as to be “1.0” in the number of used data pieces “10,000”, and to be “0.3” in the number of used data pieces “3,000”, and the like. As described above, the reference data 131 includes a set of the base sequences of the homologous chromosome 401 and the base sequences of the homologous chromosome 402. That is to say, the reference data 131 includes information indicating a genotype, such as an allele, a haplotype, and a diplotype. The reference data 131 may include a gene of a human genome indicating base sequences in tens of millions of portions, indicating base sequences of all the human genomes, or indicating SNP base sequences in the tens of millions of portions.

FIG. 5 is an illustration expressing the reference data 131 in accordance with the data density. The example in FIG. 5 shows a locus expressed in a higher concentration as the data density is higher. For instance, a base sequence for a genotype included in a region having a high concentration and denoted by the reference numeral 601 is determined with more data pieces than data pieces to be used to determine a base sequence for a genotype included in a region having a low concentration and denoted by the reference numeral 602. It is seen from this perspective that the reference data 131 has various densities depending on loci.

Next, the SNP genotype imputation will be described. Genetic data detected by the SNP microarray is data in which a part of the base sequences of one homologous chromosome and a part of the base sequences of the other homologous chromosome are decided, and a remaining part thereof is defected, like “A . . . A . . . A . . . A . . . ”, and “G . . . G . . . C . . . A . . . ”. The portion denoted by “ . . . ” represents an undecided base sequence, and the sign “A” denotes adenine, the sign “G” denotes guanine, and the sign “C” denotes cytosine. The SNP genotype imputation estimates an SNP genotype of the undecided or defected portion by using the reference data 131.

In the SNP genotype imputation, a pattern of the decided base sequence in the genetic data is compared with a pattern of the base sequence in the reference data 131, and a region of the reference data 131 where the patterns optimally match is retrieved. A base sequence in the defected portion in the genetic data is estimated from the base sequence in the reference data 131 in the retrieved region, and an SNP genotype is estimated from a result of the estimation. The result of the estimation of the genotype obtainable here is expressed by a probability, for example, “0.95” for the “AA” type, “0.44” for the “AG” type, and “0.01” for the “GG” type concerning a certain SNP.

Referring to FIG. 2, the incentive information 132 is information in which, for one or more users, a user identifier is associated with an incentive given to each user. The incentive may include data having an economic value, e.g., electronic money, a mileage point, virtual currency, a purchase point for a commodity, and a coupon, or may include data having no economic value, e.g., a certificate.

The processor 12 includes, for example, a CPU, and has an acquisition part 121, a region specifying part 122, a rarity degree calculation part 123, a contribution degree calculation part 124, an incentive calculation part 125, and an output part 126. The blocks relevant to the components included in the processor 12 are realized in response to execution of the information processing program by the CPU.

The acquisition part 121 acquires, by using the communication part 11, the genetic data transmitted from the provider terminal 2. The acquisition part 121 receives, by using the communication part 11, the attribute information transmitted from the user terminal 3. The acquisition part 121 associates the genetic data with the attribute information by using the user identifier as a key. In this manner, a dataset having the user identifier, the genetic data, the locus information, and the attribute information associated with one another is obtained.

The attribute information includes personal information of the user, residence information indicating a residence of the user, blood relation information indicating a blood relation of the user, and life pattern information indicating a life pattern of the user.

The personal information of the user includes an age, a gender, and an occupation of the user. The personal information of the user is obtainable through, for example, an input from the user to the user terminal 3. The residence information includes information indicating a name of an area where the user lives. The name of the area of the residence here includes at least one of, for example, a country name, a prefecture name, and a province or state name. The information indicating the name of the area of the residence may include information having a larger granularity than that of the prefecture, e.g., “Honshu”, “Shikoku”, “Kyushu”, and “Hokkaido” in the case of Japan, or may include information having further larger granularity than that of the country, e.g., the Asian continent, the African continent, and the Northern American continent. The residence information may be acquired by an input from the user to the user terminal 3, or may be determined on the basis of position data detected by a GPS sensor included in the user terminal 3.

The life pattern information indicates, for example, a life pattern of the user in a predetermined period (e.g., one day). Examples of the life pattern information include the average number of smoking cigarettes per day, an average alcohol intake amount per day, average consumption calories per day, the number of meals per day, meal times, an average awake time, an average bedtime, and an average sleeping time per day. The life pattern information may be input from the user, or may be monitored by a biosensor like a smartwatch.

The region specifying part 122 specifies a region where the genetic data acquired by the acquisition part 121 is located in the reference data. Here, the region specifying part 122 may specify, on the basis of the locus information associated with the genetic data, the region where the genetic data is located.

The rarity degree calculation part 123 calculates a rarity degree indicating a rarity of the genetic data on the basis of a data density associated with the region specified by the region specifying part 122. For instance, the rarity degree calculation part 123 may calculate an average value of the data densities from density data associated with all the loci in the region specified by the region specifying part 122, and may calculate a reciprocal of the calculated average value as the rarity degree. Alternatively, the rarity degree calculation part 123 may calculate an average value of the data densities associated with decided loci of base sequences in the region specified by the region specifying part 122, and may calculate a reciprocal of the calculated average value as the rarity degree. This enables calculation of the rarity degree in such a manner that a value of the rarity degree increases as the average value of the data densities in the specified region decreases.

The contribution degree calculation part 124 calculates, on the basis of the attribute information associated with the genetic data, a contribution degree of the genetic data to the genetic analysis. For instance, the contribution degree calculation part 124 determines whether the attribute information includes blood relation information, and calculates the contribution degree to be higher in determination that the blood relation information is included than in determination that the blood relation information is not included. Information specifying a blood relative of the user who provides the genetic data is, for example, adoptable as the blood relation information. Examples of the blood relative include the father, the mother, a brother, a sister, a grandfather, and other relatives. Examples of the information specifying the blood relative include an identifier of the blood relative.

In this case, the contribution degree calculation part 124 may calculate a value of the contribution degree to be higher as an information amount of the blood relation information becomes greater. For instance, the contribution degree calculation part 124 may calculate the value of the contribution degree to be higher as the number of blood relatives indicated by the blood relation information in the attribute information increases.

In the genetic analysis, the genotype of the user is compared with the genotype of the blood relative of the user to obtain an effective analysis result. In the embodiment, the contribution degree of the user is calculated to be higher as the information amount of the blood relation information becomes greater.

The contribution degree calculation part 124 may determine whether the attribution information includes the life pattern information about the user, and may calculate the contribution degree to be higher in determination that the life pattern information is included than in determination that the life pattern information is not included. In this case, the contribution degree calculation part 124 may calculate the contribution degree to be higher as the information amount of the life pattern information becomes greater. For instance, the contribution degree calculation part 124 may determine that the information amount of the life pattern information is grater as the number of data types included in the life pattern information, such as the number of smoking cigarettes per day and an alcohol intake amount per day, increases.

Alternatively, the contribution degree calculation part 124 may calculate, as a finally obtainable contribution degree, a sum of the contribution degree calculated on the basis of the blood relation information and the contribution degree calculated on the basis of the life pattern information. For instance, when the finally calculated contribution degree is defined as “B”, a contribution degree to be given in the inclusion of the blood relation information is defined as “B1”, and a contribution degree to be given in the inclusion of the life pattern information is defined as “B2”, the contribution degree calculation part 124 may calculate the contribution degree in accordance with the equation “B=B1+B2”. In this case, the value of B1 is increased as the information amount of information indicating a blood relation becomes greater, and the value of B2 is increased as the information amount of the life pattern information becomes greater.

The incentive calculation part 125 calculates an incentive to have a larger value as each of the rarity degree and the contribution degree increases. For instance, when the rarity degree is defined as “A, and the contribution degree is defined as “B”, the incentive calculation part 125 may calculate the incentive by using the following equation:

Incentive=α·A+β·B (1),

where the sign “α” denotes a weighting factor to the rarity degree, and the sign “β” denotes a weighting factor to the contribution degree. The factor α is set to a value larger than a value of the factor β when the rarity degree is given greater importance, or the factor β is set to a value larger than the factor α when the contribution degree is given greater importance.

The output part 126 outputs the incentive calculated by the incentive calculation part 125. Here, the output part 126 may register the calculated incentive in the incentive information 132 about a relevant user, and give the incentive to the user. The output part 126 may further transmit, to the user terminal 3 by using the communication part 11, offering information for offering the calculated incentive to the user.

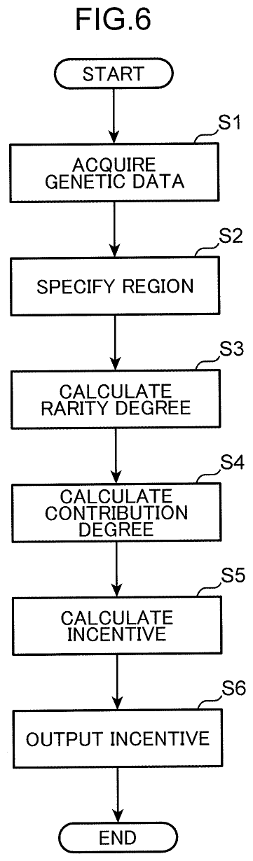

Next, a process by the information processing device 1 in the first embodiment of the disclosure will be described. FIG. 6 is a flowchart showing an example of the process by the information processing device 1 in the first embodiment of the disclosure.

In step S1, the acquisition part 121 acquires, by using the communication part 11, genetic data transmitted from the provider terminal 2.

In step S2, the region specifying part 122 specifies, on the basis of locus information associated with the genetic data, a region where the genetic data is located in the reference data 131. In the example shown in FIG. 4, a region 131a enclosed in a square is specified from the reference data 131.

In step S3, the rarity degree calculation part 123 calculates an average value of data densities in the region specified in step S2, and calculates a reciprocal of the calculated average value as a rarity degree of the genetic data. In the example in FIG. 4, the average value of the data densities of the region 131a indicates 1.3, and thus the value 1/1.3 is calculated as the rarity degree.

In step S4, the contribution degree calculation part 124 calculates a contribution degree on the basis of attribute information associated with the genetic data. In this case, the contribution degree calculation part 124 may set a value of the contribution degree to be higher as an information amount of information indicating a blood relation in the attribute information becomes greater, and may set the value of the contribution degree to be higher as an information amount of life pattern information becomes greater.

In step S5, the incentive calculation part 125 calculates an incentive in accordance with the rarity degree and the contribution degree by inputting the rarity degree calculated in step S3 and the contribution degree calculated in step S4 into the equation (1).

In step S6, the output part 126 registers the incentive calculated in step S5 in the incentive information 132 about the user having provided the genetic data, and gives the incentive to the user.

Conclusively, the information processing device 1 in the embodiment succeeds in giving a higher incentive to a user having provided genetic data having a higher rarity degree and a higher contribution degree. This results in effective collection of genetic data having a rarity and a high contribution degree to the genetic analysis.

### Second Embodiment

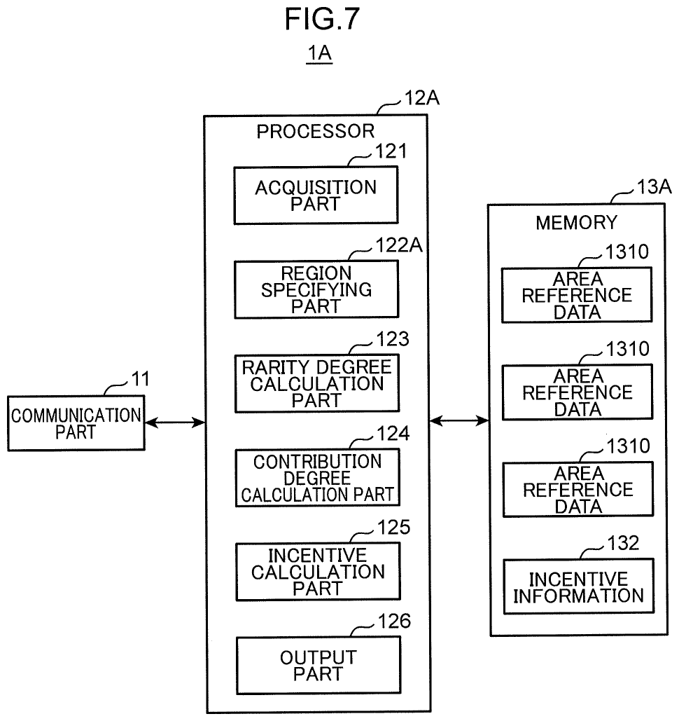

A second embodiment aims at calculating an incentive in consideration of a residence of a user. FIG. 7 is a block diagram showing an example of a configuration of an information processing device 1A in the second embodiment of the disclosure. In the embodiment, constituent elements which are the same as those in the first embodiment are given the same reference numerals and signs, and thus explanation therefor will be omitted.

A region specifying part 122A included in a processor 12A specifies, on the basis of residence information included in attribute information, area reference data 1310 for a residence of a user having provided genetic data. The region specifying part 122A then specifies a region where the genetic data is located in the specified area reference data 1310. Here, details of specifying the region are the same as those in the first embodiment, and thus the description therefor will be omitted.

A memory 13A stores three pieces of area reference data 1310 respectively for an area A, an area B, and an area C. In this case, the region specifying part 122A may determine whether the residence indicated by the residence information belongs to any one of the areas A to C, and may specify the area reference data 1310 suitable for the belonged area. The memory 13A here stores the three pieces of area reference data 1310, but this is a mere example, and the memory may store two pieces of area reference data 1310, or may store four or more pieces of area reference data 1310.

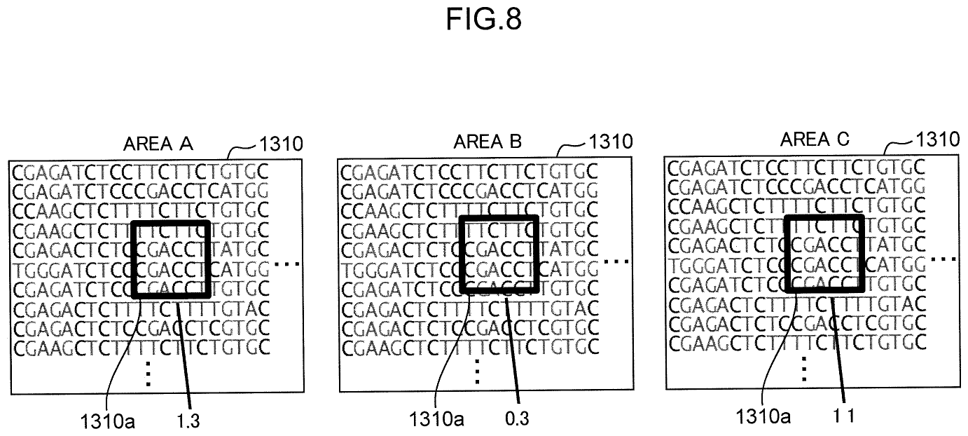

FIG. 8 illustrates an example of a data configuration of the area reference data 1310. The area reference data 1310 for the area A is generated on the basis of genetic data of a resident in the area A, the area reference data 1310 for the area B is generated on the basis of genetic data of a resident in the area B, and the area reference data 1310 for the area C is generated on the basis of genetic data of a resident in the area C. Details of the data configuration of the area reference data 1310 are the same as those of the reference data 131 except a difference in a group to be used for generating the area reference data. Specifically, the area reference data 1310 represents data in which a base sequence indicating the genotype is associated with a data density according to a locus of the base sequence.

Each of the areas A to C may have a granularity based on a country unit, an area unit constituting the country, e.g., the prefecture, or “Honshu”, “Kyushu”, and “Hokkaido” in the case of Japan, or based on a unit larger than the country unit, e.g., the Asian continent, the African continent, and the Northern American continent.

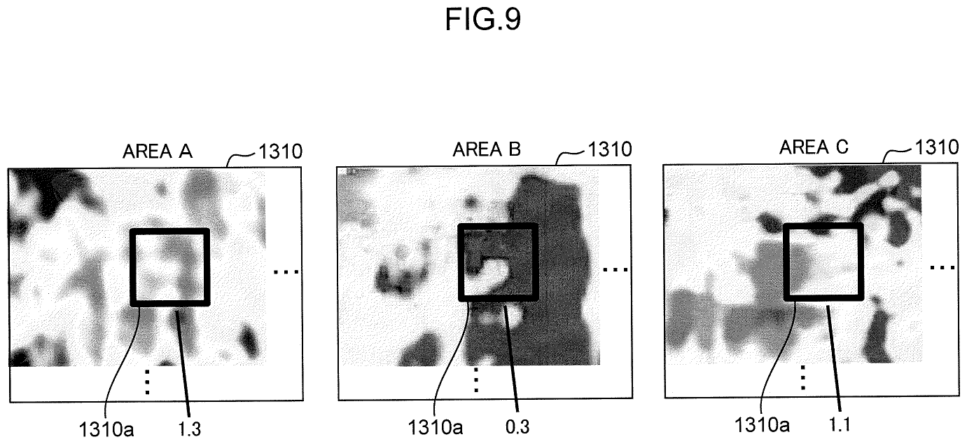

FIG. 9 is an illustration expressing the area reference data 1310 illustrated in FIG. 8 in accordance with the data density. It is seen from FIG. 9 that the data density of the area reference data 1310 differs depending on the areas A to C.

As a result of examining genotypes of thousands of people in a Japanese group, a clear difference in genotypes has been confirmed among Hokkaido area, Honshu area, Kyushu area, and Ryukyu area. It has been confirmed from this examination that the Japanese group has different genetic backgrounds among the Hokkaido area, the Honshu area, the Kyushu area, and the Ryukyu area. Thus, execution of the SNP genotype imputation using the area reference data 1310 for the residence of the user enhances an estimation accuracy of a genotype of the user. In the second embodiment, a high incentive is thus given to a user living in an area having a high rarity to effectively collect genetic data having a high rarity in each of the pieces of area reference data reference data 1310.

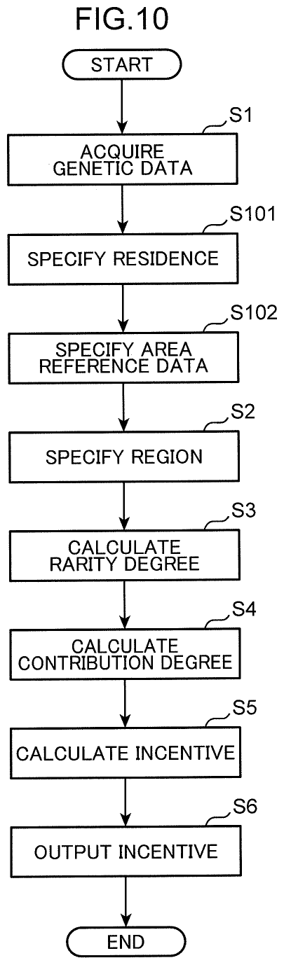

Next, a process by the information processing device 1A in the second embodiment of the disclosure will be described. FIG. 10 is a flowchart showing an example of the process by the information processing device 1A in the second embodiment of the disclosure. In the flowchart shown in FIG. 10, steps which are the same as those in FIG. 6 are given the same reference numerals and signs, and thus explanation therefor will be omitted.

In step S101 subsequent to step S1, the region specifying part 122A specifies a residence of a user having provided genetic data acquired in step S1 from residence information included in attribute information associated with the genetic data.

In step S102, the region specifying part 122A specifies area reference data 1310 for the residence specified in step S101. Thereafter, an incentive to be given to the user is calculated and output by using the specified area reference data 1310 and the genetic data acquired in step S1.

Referring to the left view in FIG. 8, when the residence of the user belongs to the area A, the area reference data 1310 for the area A is specified and a region 1310a where the genetic data is located in the specified area reference data 1310 is specified. An average value of data densities in the region 1310a indicates 1.3 here, and a rarity degree is calculated to be 1/1.3.

Referring to the middle view in FIG. 8, when the residence of the user belongs to the area B, a region 1310a where the genetic data is located in the area reference data 1310 for the area B is specified. An average value of data densities in the region 1310a indicates 0.3 here, and thus a rarity degree is calculated to be 1/0.3.

In the example in FIG. 8, the average value of the data densities of the region 1310a is larger in order of the area A, the area C, and the area B. Hence, the rarity is higher in order of the area B, the area C, and the area A. As a result, an incentive to be given to the user belonging to the area B is the maximum, and an incentive to be given to the user belonging to the area A is the minimum.

Conclusively, the information processing device 1A in the second embodiment succeeds in giving a high incentive to a user in a residence belonging to an area corresponding to the area reference data 1310 having a low data density. This thus motivates the user in the residence belonging to the area corresponding to the area reference data 1310 having the low data density to provide genetic data, resulting in effective collection of the genetic data.

This disclosure can adopt modifications described below.

(1) Although the region specifying part 122 specifies the region 131a by using locus information associated with genetic data, this disclosure is not limited thereto. For instance, the region specifying part 122 may compare a pattern of a base sequence of the genetic data with a pattern of a base sequence in the reference data 131, retrieve a region of the reference data 131 where the patterns optimally match, and specify the retrieved region as the region 131a where the genetic data is located. This is applicable to the region specifying part 122A in the same manner.

(2) Although the information processing device 1 stores the incentive information 132, this disclosure is not limited thereto. For instance, an external server owned by a manager who manages an incentive may store the incentive information 132. When the incentive indicates electronic money, a financial institution serves as the manager for example. When the incentive indicates a mileage point, an airline company serves as the manager for example. When the incentive indicates a point given in response to purchase of a commodity, a point running company serves as the manager for example.

(3) In the first embodiment, the incentive calculation part 125 may calculate an incentive only on the basis of a rarity degree. In this case, the contribution degree calculation part 124 is excludable.

(4) Although the information processing device 1 stores the reference data 131, this disclosure is not limited thereto, and an external server may store the reference data.

### INDUSTRIAL APPLICABILITY

This disclosure achieves effective collection of genetic data having a rarity, and thus is useful in the genetic industry.

1. An information processing method for an information processing device that performs an information process by using reference data, the information processing method comprising:
acquiring genetic data detected by a gene detector and including a base sequence indicating a genotype of a user;
specifying a region where the genetic data is located in the reference data, the reference data being data that in which a base sequence indicating a genotype of a genome is associated in advance with a data density according to a locus of the base sequence;
calculating, on the basis of the data density associated with the specified region, a rarity degree indicating a rarity of the genetic data;
calculating an incentive to be given to the user in accordance with the calculated rarity degree; and
outputting the calculated incentive.

2. The information processing method according to claim 1, wherein the genetic data is associated with attribute information including an attribute of the user,
the information processing method further comprising:
calculating, on the basis of the attribute information, a contribution degree of the genetic data to a genetic analysis, wherein,
in the calculating of the incentive, the incentive is calculated in accordance with the rarity degree and the contribution degree.

3. The information processing method according to claim 1, wherein the genetic data is associated with locus information indicating a locus of the base sequence indicating the genotype, and,
in the calculating of the rarity degree, the region where the genetic data is located in the reference data is specified on the basis of the locus information.

4. The information processing method according to claim 2, wherein the attribute information includes information indicating a residence of the user,
the reference data includes a plurality of pieces of area reference data respectively for predetermined areas, and,
in the specifying of the region, the region where the genetic data is located in area reference data corresponding to the information about the residence is specified.

5. The information processing method according to claim 2, wherein,
in the calculating of the contribution degree, the attribute information is determined whether to include information indicating a blood relation of the user, and the contribution degree is calculated to be higher in determination that the information indicating the blood relation is included than in determination that the information indicating the blood relation is not included.

6. The information processing method according to claim 5, wherein,
in the calculating of the contribution degree, the contribution degree is calculated to be higher as an information amount of the information indicating the blood relation in the attribute information becomes greater.

7. The information processing method according to claim 2, wherein,
in the calculating of the contribution degree, the attribute information is determined whether to include information indicating a life pattern of the user, and the contribution degree is calculated to be higher in determination that the information indicating the life pattern is included than in determination that the information indicating the life pattern is not included.

8. The information processing method according to claim 7, wherein,
in the calculating of the contribution degree, the contribution degree is calculated to be higher as an information amount of the information indicating the life pattern of the user in the attribute information becomes greater.

9. An information processing device that performs an information process by using reference data, the information processing device comprising:
an acquisition part that acquires genetic data detected by a gene detector and including a base sequence indicating a genotype of a user;
a region specifying part that specifies a region where the genetic data is located in the reference data, the reference data being data that in which a base sequence indicating a genotype of a genome is associated in advance with a data density according to a locus of the base sequence;
a rarity degree calculation part that calculates a rarity degree indicating a rarity of the genetic data on the basis of the data density associated with the region specified by the region specifying part;
an incentive calculation part that calculates an incentive to be given to the user in accordance with the rarity degree calculated by the rarity degree calculation part; and
an output part that outputs the incentive calculated by the incentive calculation part.

10. A non-transitory computer readable recording medium storing an information processing program causing a computer to serve as an information processing device that performs an information process by using reference data, the information processing program comprising:
further causing the computer to serve as:
an acquisition part that acquires genetic data detected by a gene detector and including a base sequence indicating a genotype of a user;
a region specifying part that specifies a region where the genetic data is located in the reference data, the reference data being data that in which a base sequence indicating a genotype of a genome is associated in advance with a data density according to a locus of the base sequence;
a rarity degree calculation part that calculates a rarity degree indicating a rarity of the genetic data on the basis of the data density associated with the region specified by the region specifying part;
an incentive calculation part that calculates an incentive to be given to the user in accordance with the rarity degree calculated by the rarity degree calculation part; and
an output part that outputs the incentive calculated by the incentive calculation part.
